(19) United States
(12) Patent Application Publication
(10) Pub. No.: US 2024/0027322 A1
(43) Pub. Date: Jan. 25, 2024

(54) DETECTION METHOD AND DETECTION SYSTEM

(71) Applicant: Panasonic Intellectual Property Management Co., Ltd., Osaka (JP)

(72) Inventor: TAKASHI KANNO, Osaka (JP)

(21) Appl. No.: 18/480,536

(22) Filed: Oct. 4, 2023

Related U.S. Application Data
(63) Continuation of application No. PCT/JP2022/017375, filed on Apr. 8, 2022.

(30) Foreign Application Priority Data
Apr. 23, 2021 (JP) 2021-073680

(51) Int. Cl. G01N 15/14; G01N 27/447

(52) U.S. Cl. CPC G01N 15/1404; G01N 15/1456; G01N 27/44786; G01N 27/44721; G01N 2015/1415

(57) ABSTRACT

A detection method includes applying a magnetic field to a sample including a composite particle, an unbound particle, and a first solvent, thereby retaining the composite particle and the unbound particle, each of the composite particle and the unbound particle including a magnetic dielectric particle modified by a substance capable of specifically binding to a target substance, the composite particle being bound to the target substance, the unbound particle being not bound to the target substance; replacing, when a predetermined condition is satisfied, at least part of the first solvent with a second solvent with lower electrical conductivity than the first solvent in a state in which the composite particle and the unbound particle are retained; stopping the application of the magnetic field and applying an electric field, thereby separating the composite particle and the unbound particle by dielectrophoresis; and detecting the separated composite particle, thereby detecting the target substance.

### BACKGROUND

### 1. Technical Field

The present disclosure relates to a detection method and a detection system for detecting a target substance, for example, a virus.

### 2. Description of the Related Art

Japanese Unexamined Patent Application Publication No. 2012-071256 discloses a minute object collection device for collecting, from a test solution including a minute object, the minute object. The disclosed minute object collection device includes a minute object collector, a test solution feeder, and a separation solution feeder. The minute object collector collects the minute object by applying an AC voltage of a first frequency or an AC voltage of a second frequency to a collection electrode. The test solution feeder introduces the test solution into the minute object collector. The separation solution feeder introduces a separation solution into the minute object collector.

### SUMMARY

One non-limiting and exemplary embodiment provides a detection method and so on which can easily improve detection accuracy of a target substance.

In one general aspect, the techniques disclosed here feature a detection method including applying a magnetic field to a sample including a composite particle, an unbound particle, and a first solvent, thereby retaining the composite particle and the unbound particle, each of the composite particle and the unbound particle including a dielectric particle that has magnetism and is modified by a substance capable of specifically binding to a target substance, the composite particle being bound to the target substance, the unbound particle being not bound to the target substance; replacing, when a predetermined condition is satisfied, at least part of the first solvent with a second solvent with lower electrical conductivity than the first solvent in a state in which the composite particle and the unbound particle are retained; stopping the application of the magnetic field and applying an electric field, thereby separating the composite particle and the unbound particle by dielectrophoresis; and detecting the separated composite particle, thereby detecting the target substance.

The detection method according to one aspect of the present disclosure and so on are advantageous in that the detection accuracy of the target substance can be easily improved.

It should be noted that general or specific embodiments of the present disclosure may be implemented as a device, a system, a method, an integrated circuit, a computer program, a computer-readable recording medium, or any selective combination thereof. The computer-readable recording medium includes a nonvolatile recording medium, such as a CD-ROM (Compact Disc-Read Only Memory).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a perspective view illustrating a schematic configuration of a detection system according to an embodiment;

FIG. 1B is an explanatory view of particle species and so on in the embodiment;

FIG. 2 represents a block diagram illustrating the schematic configuration of the detection system according to the embodiment along with a sectional view of a separator;

FIG. 3 is a plan view illustrating a configuration of an electrode set in the embodiment;

FIG. 4 is a correlation graph between a cross-frequency and electrical conductivity of a sample for each of a composite particle and an unbound particle in the embodiment;

FIG. 5 is an explanatory view representing retention of the composite particle and the unbound particle by a magnetic field applicator in the embodiment;

FIG. 6 is an explanatory view representing replacement of a first solvent with a second solvent by an exchanger in the embodiment; and

FIG. 7 is a flowchart representing an example of a detection method according to the embodiment.

### DETAILED DESCRIPTIONS

Embodiments of the present disclosure will be described below with reference to the drawings.

It is to be noted that any of embodiments described below represent a general or specific example. Numerical values, shapes, materials, constituent elements, layout positions of and connection forms between the constituent elements, steps, order of the steps, etc., which are described in the following embodiments, are merely illustrative, and they are not purported to limit the scope of the present disclosure.

The drawings are not always exactly drawn in a strict sense. In the drawings, substantially the same constituent elements are denoted by the same reference signs, and duplicate description of those constituent elements is omitted or simplified in some cases.

In the following description, terms indicating positional relationships between elements, such as “parallel” and “perpendicular”, words indicating the shapes of elements, such as “rectangular”, and a numerical range do not always indicate the meanings as per intended in a strict sense. Those terms and so on should be interpreted as indicating substantially equivalent meanings and range and as allowing a slight tolerance, for example, a difference of several percentages.

In the following description, detecting a target substance includes, in addition to finding the target substance and confirming the presence of the target substance, measuring a quantity (for example, number, concentration, or the like) of the target substance or a range of the quantity.

### Embodiments

A detection method and a detection system according to embodiments are a method and a system for separating a composite particle and an unbound particle in a liquid by dielectrophoresis (DEP) and for detecting a target substance included in the separated composite particle.

Here, the dielectrophoresis is a phenomenon that a force acts on a dielectric particle put under a non-uniform electric field. There is no need of charging the particle to generate the above force.

The target substance is a substance to be detected and is, for example, a molecule of pathogenic protein or the like, a virus (such as outer protein), or a cell (such as polysaccharide). The target substance is also called an analyte or a detection target in some cases.

The detection method and the detection system for detecting the target substance by the dielectrophoresis will be described in detail below with reference to the drawings.

### Configuration of Detection System

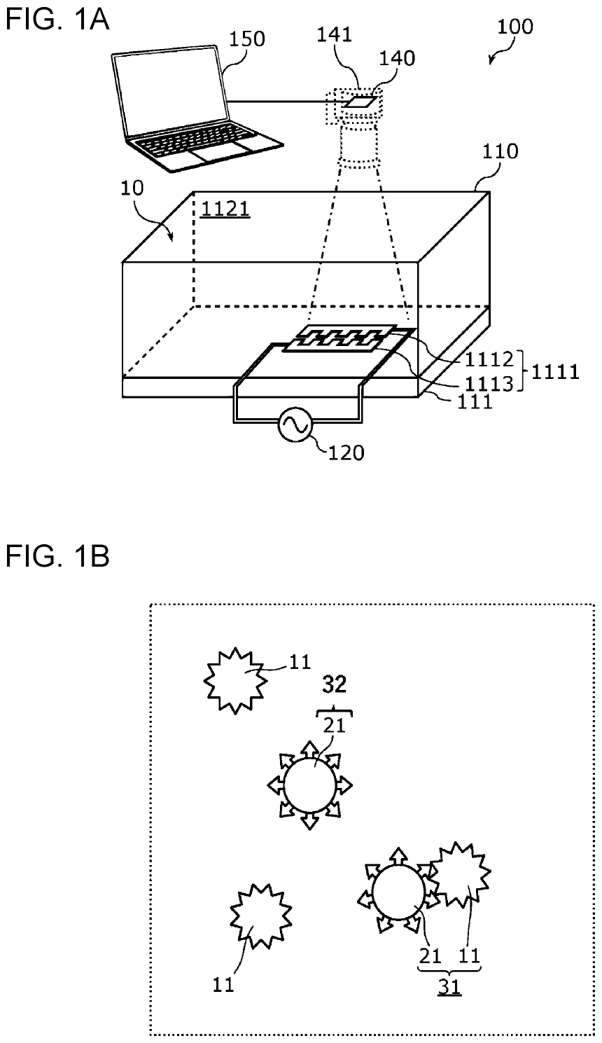

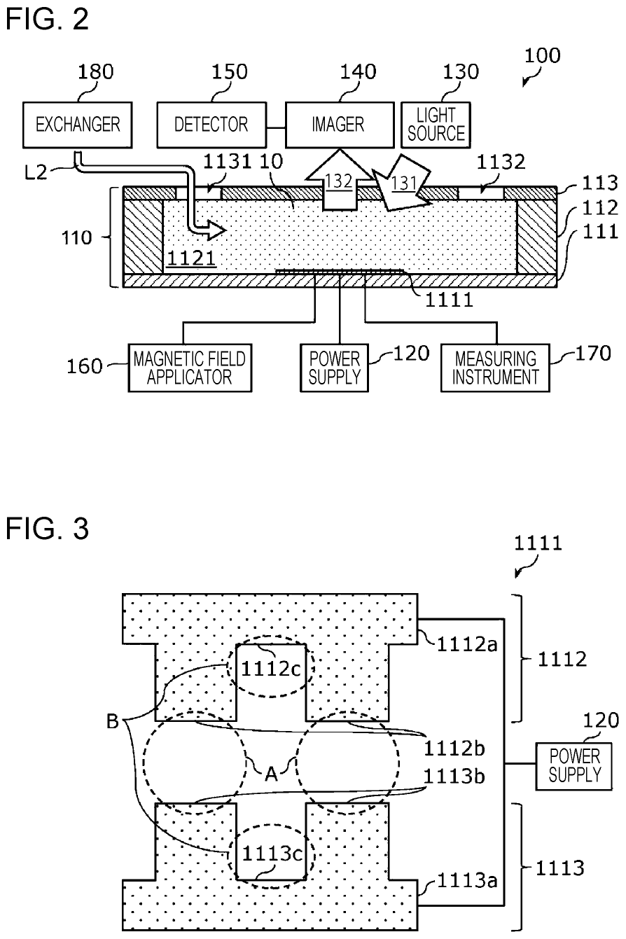

First, a configuration of a detection system 100 is described with reference to FIGS. 1A, 1B, and FIG. 2. FIG. 1A is a perspective view illustrating the schematic configuration of the detection system 100 according to the embodiment. FIG. 1B is an explanatory view of particle species and so on in the embodiment. FIG. 2 represents a block diagram illustrating the schematic configuration of the detection system 100 according to the embodiment along with a sectional view of a separator 110. In FIG. 1A, the separator 110 is illustrated in the form just representing a contour to be able to see the inside of the separator 110. FIG. 1A is referenced to explain a relationship among constituent elements illustrated. It is to be noted that FIG. 1A does not restrict layout positions, layout directions, postures, and so on of the individual constituent elements when the detection system 100 is used. FIG. 1B illustrates (i) a composite particle 31 that is a particle species formed by binding of a target substance 11 and a dielectric particle 21, (ii) an unbound particle 32 that is the dielectric particle 21 not bound to the target substance 11, and (iii) the target substance 11 not bound to the dielectric particle 21, those particles 31 and 32 and the target substance 11 being included in a sample 10 that is put in a space 1121 illustrated in FIG. 1A. FIG. 2 represents the block diagram illustrating the individual constituent elements of the detection system 100 along with the sectional view of the separator 110, illustrated in FIG. 1A, cut along a direction parallel to the drawing sheet. Note that some of the constituent elements of the separator 110 in FIG. 2 are illustrated in FIG. 1A in the form not having thicknesses.

As illustrated in FIGS. 1A and 2, the detection system 100 includes the separator 110, a power supply 120, a light source 130, an imager 140, a detector 150, a magnetic field applicator 160, a measuring instrument 170, and an exchanger 180.

The separator 110 is a container for containing the sample 10 that may include the target substance 11, and it has a space 1121 therein. The sample 10 is put into the space 1121. The separator 110 separates, inside the space 1121, the composite particle 31 and the unbound particle 32 in a liquid (namely, a solvent included in the sample 10) by the dielectrophoresis. The separator 110 spatially separates the composite particle 31 and the unbound particle 32 into different positions. The sample 10 is a liquid. The sample 10 includes a first solvent L1 and the unbound particle 32. When the analyte includes the target substance 11, the sample 10 further includes the composite particle 31 that is formed by the target substance 11 and the dielectric particle 21. Stated another way, the sample 10 includes the first solvent L1, the composite particle 31, and the unbound particle 32. Contaminants are mixed in the sample 10 in some cases. As described later, the unbound particle 32 is the dielectric particle 21 not bound to the target substance 11.

As illustrated in FIG. 1B, the composite particle 31 is a particle resulting from binding of the target substance 11 and the dielectric particle 21 that has magnetism and is modified by a substance with a property of specifically binding to the target substance 11. Thus, in the composite particle 31, the target substance 11 and the dielectric particle 21 are bound to each other while the substance with the property of specifically binding to the target substance 11 is interposed therebetween.

The dielectric particle 21 is a particle that has magnetism to be attracted by a magnet and that can be polarized with application of an electric field. The dielectric particle 21 may contain, for example, a fluorescent substance. In this case, by applying, from the light source 130 (described later), irradiation light of a wavelength at which the fluorescent substance is excited, the dielectric particle 21 can be detected by detecting light in a wavelength band of fluorescence emission. The dielectric particle 21 is not limited to the particle containing the fluorescent substance. For example, a polystyrene particle, a glass particle, or the like not containing the fluorescent substance may be used as the dielectric particle 21. The dielectric particle 21 may have magnetism with a magnetic particle embedded therein. More specifically, the dielectric particle 21 may have ferromagnetism with a magnetic substance (magnetic particle), such as ferrite, embedded therein.

Here, the substance with the property of specifically binding to the target substance 11 is a substance capable of specifically binding to the target substance 11 and is also called a specifically binding substance. A combination of the specifically binding substance with the target substance 11 is given by, for example, antibody with antigen, enzyme with substrate or coenzyme, receptor with hormone, protein A or protein G with antibody, avidins with biotin, calmodulin with calcium, lectin with saccharide, or a tag binding substance, such as nickel—nitrilotriacetate or glutathione, with peptide tag, such as 6×histidine or glutathione S-transferase.

The unbound particle 32 is the dielectric particle 21 not forming the composite particle 31. Thus, the unbound particle 32 is the dielectric particle 21 not bound to the target substance 11. The unbound particle 32 is also called a free (F) component. On the other hand, the dielectric particle 21 and the specifically binding substance each included in the composite particle 31 are also called bound (B) components.

An inner structure of the separator 110 will be described below. As illustrated in FIG. 2, the separator 110 includes a first substrate 111, a spacer 112, and a second substrate 113.

The first substrate 111 is, for example, a sheet made of glass or resin. The first substrate 111 has an upper surface defining the bottom of the space 1121, and an electrode set 1111 to which an AC voltage is applied from the power supply 120 is formed on the upper surface. The electrode set 1111 includes a first electrode 1112 and a second electrode 1113 as illustrated in FIG. 1A and can generate a non-uniform electric field (also called an electric field gradient) on the first substrate 111. Thus, the electrode set 1111 is an example of an electric field gradient generator for generating (or forming) the electric field gradient. Details of the electrode set 1111 will be described later with reference to FIG. 3.

The spacer 112 is disposed on the first substrate 111. A through-hole corresponding to the shape of the space 1121 is formed to penetrate the spacer 112. Stated another way, the space 1121 is formed by the through-hole sandwiched between the first substrate 111 and the second substrate 113. As described above, the sample 10 that may include the composite particle 31 and the unbound particle 32 is introduced to the space 1121. The spacer 112 serves as an outer wall surrounding the through-hole and has an inner surface defining the space 1121. The spacer 112 is made of, for example, a resin material with high adhesion to both the first substrate 111 and the second substrate 113.

The second substrate 113 is, for example, a transparent sheet made of glass or resin and is disposed on the spacer 112. For example, a polycarbonate substrate can be used as the second substrate 113. A supply hole 1131 and a discharge hole 1132 each in fluid communication with the space 1121 are formed in the second substrate 113 to penetrate therethrough. The sample 10 is supplied to the space 1121 via the supply hole 1131 and is discharged from the space 1121 via the discharge hole 1132. The separator 110 may be constituted without disposing the second substrate 113. Thus, the second substrate 113 is not an essential constituent element. In an example, the space 1121 enabling the separator 110 to function as a container may be formed by the first substrate 111 and the spacer 112 defining respectively the bottom and the inner surface of the space 1121.

The power supply 120 is an AC power supply and applies the AC voltage to the electrode set 1111 on the first substrate 111. The power supply 120 may be any type of power supply as far as being able to supply the AC voltage and is not limited to a particular one. The AC voltage may be supplied from an external power supply. In such a case, the power supply 120 may not need to be included in the detection system 100.

The light source 130 applies irradiation light 131 to the sample 10 in the space 1121. The irradiation light 131 is applied to the sample 10 through the transparent second substrate 113. Detection light 132 is generated from the sample 10 corresponding to the irradiation light 131, and the dielectric particle 21 included in the sample 10 is detected by detecting the detection light 132. For example, when the fluorescent substance is contained in the dielectric particle 21 as described above, the fluorescent substance contained in the dielectric particle 21 is excited upon the irradiation light 131 being applied as excitation light, and fluorescence emitted from the fluorescent substance is detected as the detection light 132.

The light source 130 may be a light source using the known technique. For example, a laser, such as a semiconductor laser or a gas laser, can be used as the light source 130. The wavelength of the irradiation light 131 emitted from the light source 130 is set to a wavelength at which the interaction with any material contained in the target substance 11 is small. For example, when the target substance 11 is a virus, the irradiation light 131 of the wavelength longer than or equal to 400 nm and shorter than or equal to 2000 nm is selected. The wavelength of the irradiation light 131 may be given as a wavelength (for example, longer than or equal to 600 nm and shorter than or equal to 850 nm) that is available by the semiconductor laser.

Note that the light source 130 may not need to be included in the detection system 100. For example, when the dielectric particle 21 has a large size, the detection light can be observed by combining optical elements, such as lenses, without utilizing luminous phenomenon such as the fluorescence emission. In other words, the fluorescent substance may not need to be contained in the dielectric particle 21. In such a case, the irradiation light 131 may not need to be emitted from the light source 130. Thus, in that case, the dielectric particle 21 can be detected by utilizing, instead of the light source 130, outside light emitted from the sun, a fluorescent lamp, or the like.

The imager 140 is, for example, a CMOS image sensor or a CCD image sensor. By receiving the detection light 132 generated from the sample 10, the imager 140 generates and outputs an image. In an example, the imager 140 is built in a camera 141 or the like, is horizontally disposed on the surface of the first substrate 111, and takes an image of a location corresponding to the electrode set 1111 through an optical element (not illustrated), such as a lens, included in the camera 141. Thus, the imager 140 is used to take an image of the composite particle 31 separated from the unbound particle 32 by the separator 110 and to detect the target substance 11 included in the composite particle 31.

In the example in which the dielectric particle 21 contains the fluorescent substance, the imager 140 takes an image of the fluorescence emitted from the fluorescent substance contained in the dielectric particle 21. The detection system 100 may include a photodetector instead of the imager 140. In that case, the photodetector may detect the detection light 132, such as the fluorescence, from a region on the first substrate 111 where the composite particle 31 separated by the dielectrophoresis tends to gather. When the photodetector is used instead of the imager 140 as described above, the detector 150 may detect the target substance 11 bound to the dielectric particle 21 based on the intensity of the detection light 132.

The detection system 100 may include an optical lens or an optical filter between the light source 130 and the separator 110 or between the separator 110 and the imager 140. For example, a long-pass filter capable of cutting off the irradiation light 131 from the light source 130 and allowing the detection light 132 to pass therethrough may be disposed between the separator 110 and the imager 140.

The detector 150 obtains an image output from the imager 140 and detects the dielectric particle 21 included in the sample 10 based on the obtained image. The detection system 100 according to the embodiment can separately count the composite particle 31 and the unbound particle 32. In other words, the dielectric particle 21 forming the composite particle 31 and the dielectric particle 21 forming the unbound particle 32 can be detected in a distinguishable way. Accordingly, the detector 150 detects the target substance 11 included in the composite particle 31 in the sample 10 by detecting the dielectric particle 21 based on the image.

In an example, the detector 150 detects the dielectric particle 21 by comparing an i-th (i=1 to n) pixel value of an i-th pixel in a reference image not including the dielectric particle 21, the reference image being taken in advance, and an i-th pixel value of an i-th pixel in an image including the dielectric particle 21 (namely, an image taken for the sample 10). More specifically, a pixel j (1≤j≤n) for which the pixel value in the image including the dielectric particle 21 is greater than a predetermined value is extracted. Furthermore, if the pixel value of a pixel in the reference image, corresponding to the pixel j, is smaller than a predetermined value, the pixel j is determined to be the pixel corresponding to the dielectric particle 21. A dielectric particle p and a dielectric particle q (p≠q) can be distinguished based on not only the fact that pixels corresponding to the dielectric particles p and pixels corresponding to the dielectric particles q are distributed discontinuously, but also the number of pixels occupied by one dielectric particle and the outer shape of the pixels of one dielectric particle.

In such a manner, a detection result of the composite particle 31 in the sample 10 is obtained by the detector 150.

The detector 150 is realized, for example, by executing a program to perform the above-described image analysis with a circuit, such as a processor, and a storage device, such as a memory. Alternatively, the detector 150 may be realized with a dedicated circuit. In an example, the detector 150 is incorporated in a computer.

The magnetic field applicator 160 retains the magnetic dielectric particle 21 (namely, the composite particle 31 and the unbound particle 32) in the vicinity of the electrode set 1111 by applying a magnetic field 161 to the sample 10 of which solvent is the first solvent L1 (see FIG. 5). FIG. 5 is an explanatory view representing retention of the composite particle 31 and the unbound particle 32 by the magnetic field applicator 160 in the embodiment.

The word “retain (retention)” used here indicates that the dielectric particle 21 is attracted to a place where the magnetic field 161 is applied, thereby causing the dielectric particle 21 to stay at the place during a period in which the magnetic field 161 is applied. In the embodiment, the dielectric particle 21 is to be retained just to such an extent that the dielectric particle 21 will not be swept away when the first solvent L1 is replaced with the second solvent L2 by the exchanger 180 (described later) during the period in which the magnetic field 161 is applied.

The magnetic field applicator 160 is disposed, for example, under the first substrate 111 outside the separator 110. In the embodiment, the magnetic field applicator 160 is disposed to apply the magnetic field 161 to the sample 10 at a place opposite to the electrode set 1111. Stated another way, at a position of the electrode set 1111 (electrode) for applying an electric field to the sample 10, the magnetic field applicator 160 applies the magnetic field 161 to the sample 10 of which solvent is the first solvent L1.

The magnetic field applicator 160 may be formed by an electromagnet or a permanent magnet. When the magnetic field applicator 160 is formed by the electromagnet, application and release of the magnetic field 161 can be controlled by adjusting a current supplied to flow through a coil. On the other hand, when the magnetic field applicator 160 is formed by the permanent magnet, the application of the magnetic field 161 can be released by inserting a magnetic shield member between the permanent magnet and the electrode set 1111 or by moving the permanent magnet away from the electrode set 1111 with an actuator, for example.

The measuring instrument 170 applies an AC voltage between a pair of electrodes disposed to position in the sample 10 of which solvent is the first solvent L1, and measures, based on a current flowing between the pair of electrodes, electrical conductivity of the sample 10 of which solvent is the first solvent L1. In the embodiment, the measuring instrument 170 applies the AC voltage between the first electrode 1112 and the second electrode 1113 of the electrode set 1111 (described later) from the power supply 120, thereby generating the current between the first electrode 1112 and the second electrode 1113. Alternatively, the measuring instrument 170 may measure the electrical conductivity of the sample 10 by using a pair of electrodes other than the electrode set 1111 and a power supply other than the power supply 120.

The exchanger 180 replaces at least part of the first solvent L1 with the second solvent L2 with lower electrical conductivity than the first solvent L1 when a predetermined condition is satisfied. The second solvent L2 is pure water or a solvent containing a small quantity of ions and so on, for example, and is a solvent with electrical conductivity of lower than 0.1 S/m (the electrical conductivity of the second solvent L2 may be lower than 0.03 S/m). In an example, the second solvent L2 is an aqueous solution obtained by diluting physiological saline or a phosphate buffer with pure water, or an aqueous solution containing salt such as sodium chloride, potassium chloride, or potassium phosphate.

In the embodiment, the predetermined condition is that the electrical conductivity of the sample 10 of which solvent is the first solvent L1 is higher than or equal to a predetermined value. More specifically, when the electrical conductivity of the sample 10 of which solvent is the first solvent L1, measured by the measuring instrument 170, is higher than or equal to the predetermined value, the exchanger 180 replaces at least part of the first solvent L1 with the second solvent L2. On the other hand, when the electrical conductivity of the sample 10 of which solvent is the first solvent L1, measured by the measuring instrument 170, is lower than the predetermined value, the exchanger 180 does not execute the replacement of the solvent. The predetermined value is, for example, 0.1 S/m (or may be 0.03 S/m).

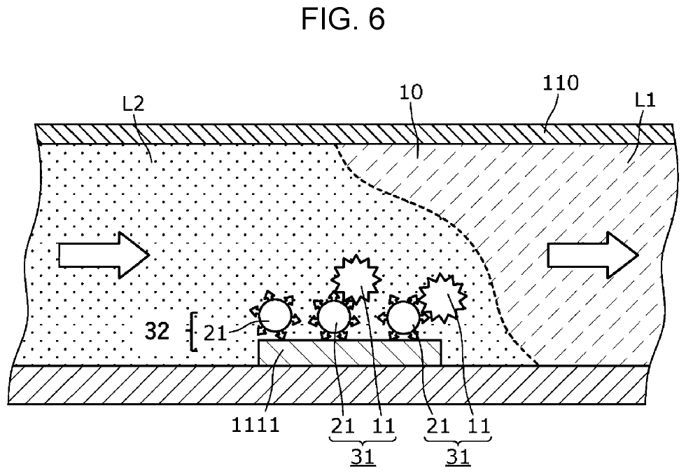

In the embodiment, the exchanger 180 replaces the first solvent L1 in the space 1121 with the second solvent L2 by, as illustrated in FIG. 6, introducing the second solvent L2 to sweep away the first solvent L1. FIG. 6 is an explanatory view representing the replacement of the first solvent L1 with the second solvent L2 by the exchanger 180 in the embodiment. More specifically, the exchanger 180 includes a supply source of the second solvent L2, a flow path connecting the supply source of the second solvent L2 and the supply hole 1131 (namely, a flow path through which the second solvent L2 flows), and a valve disposed in the flow path. When the predetermined condition is satisfied, the exchanger 180 opens the valve and supplies the second solvent L2 into the space 1121 through the supply hole 1131. Thus, the first solvent L1 in the space 1121 is swept away by the second solvent L2, and the first solvent L1 is discharged to the outside of the space 1121 through the discharge hole 1132. As a result, the first solvent L1 in the space 1121 is replaced with the second solvent L2.

### Shape and Arrangement of Electrode Set

The shape and the arrangement of the electrode set 1111 on the first substrate 111 will be described below with reference to FIG. 3. FIG. 3 is a plan view illustrating a configuration of the electrode set 1111 in the embodiment. FIG. 3 illustrates the configuration of the electrode set 1111 in a plan view when viewed from a side including the imager 140. Note that FIG. 3 illustrates a schematic configuration of part of the electrode set 1111 for the sake of simplification.

As described above, the electrode set 1111 includes the first electrode 1112 and the second electrode 1113 that are disposed on the first substrate 111. The first electrode 1112 and the second electrode 1113 are each electrically connected to the power supply 120.

The first electrode 1112 includes a first base 1112a extending in a first direction (a left-right direction on the drawing sheet of FIG. 3) and two first protrusions 1112b protruding from the first base 1112a in a second direction (an up-down direction on the drawing sheet of FIG. 3) intersecting the first direction. A first recess 1112c is formed between the two first protrusions 1112b. The two first protrusions 1112b are arranged to face the second electrode 1113 (specifically, two second protrusions 1113b described later). In the two first protrusions 1112b and the first recess 1112c, a length in the first direction and a length in the second direction are each, for example, about 5 micrometers. The length of each of the two first protrusions 1112b and the first recess 1112c in the first direction is not limited to about 5 micrometers. The length of each of the two first protrusions 1112b and the first recess 1112c in the second direction is not limited to about 5 micrometers.

A shape and a size of the second electrode 1113 are substantially the same as the shape and the size of the first electrode 1112. In more detail, the second electrode 1113 also includes a second base 1113a extending in the first direction (the left-right direction on the drawing sheet of FIG. 3) and two second protrusions 1113b protruding from the second base 1113a in the second direction (the up-down direction on the drawing sheet of FIG. 3) intersecting the first direction. A second recess 1113c is formed between the two second protrusions 1113b. The two second protrusions 1113b are arranged to face the first electrode 1112 (specifically, the two first protrusions 1112b).

With the application of the AC voltages to the first electrode 1112 and the second electrode 1113 described above, the non-uniform electric field is generated on the first substrate 111. The AC voltage applied to the first electrode 1112 and the AC voltage applied to the second electrode 1113 may be substantially the same or may have a phase difference therebetween. The phase difference between the applied AC voltages may be set to, for example, 180 degrees.

The position of the electrode set 1111 is not limited to a place on the first substrate 111. The electrode set 1111 is just to be disposed in the vicinity of the sample 10 inside the space 1121. Here, the wording “the vicinity of the sample 10” indicates a range around the sample 10 within which the electric field can be generated in the sample 10 by the AC voltage applied to the electrode set 1111. In other words, the electrode set 1111 may be in direct contact with the sample 10 inside the space 1121 or may form the electric field in a region including the sample 10 from the outside of the space 1121.

### Distribution of Electric field Strength on First Substrate

A distribution of electric field strength of the non-uniform electric field generated on the first substrate 111 will be described below with reference to FIG. 3.

As illustrated in FIG. 3, the non-uniform electric field forms, on the first substrate 111, a first electric field region A where the electric field strength is relatively high and a second electric field region B where the electric field strength is relatively low. The first electric field region A is a region where the electric field strength is higher than that in the second electric field region B, the region being positioned between the first protrusion 1112b and the second protrusion 1113b facing each other.

The electric field strength depends on an electrode-to-electrode distance between a pair of electrodes generating an electric field. The electric field strength becomes lower as the electrode-to-electrode distance increases and becomes higher as the electrode-to-electrode distance decreases. A position at which respective ends of the first protrusion 1112b and the second protrusion 1113b in the first direction face each other is a position in the electrode set 1111 at which the distance between the first electrode 1112 and the second electrode 1113 is minimum and at which the electric field strength is maximized. The first electric field region A is a region of predetermined size including the above-mentioned position at which the distance between the first electrode 1112 and the second electrode 1113 is minimum.

The second electric field region B is a region where the electric field strength is lower than that in the first electric field region A, the region being formed between the first recess 1112c and the second recess 1113c facing each other. In the above-mentioned region, the distance between the first electrode 1112 and the second electrode 1113 is maximized. More specifically, the electric field strength becomes lower at a position closer to the bottom of the first recess 1112c or the second recess 1113c. The second electric field region B is a region including the bottom of the first recess 1112c or the second recess 1113c at which the electric field strength is particularly low.

### Positive Precipitation and Negative Precipitation by Dielectrophoresis

Positive precipitation and negative precipitation of the dielectric particle in the case of using the electrode set 1111 constituted as described above will be described below with reference to FIG. 3. Depending on a frequency of the AC voltage applied to the electrode set 1111, ion species in a liquid around the dielectric particle 21, and so on, the dielectric particle 21 accumulates in the first electric field region A where the electric field strength is relatively high or the second electric field region B where the electric field strength is relatively low. On that occasion, the behavior of the dielectric particle during the dielectrophoresis (namely which one of the positive precipitation and the negative precipitation occurs) is determined depending on a real part of the Claudius-Mosotti coefficient. When the real part of the Claudius-Mosotti coefficient takes a positive numerical value based on various conditions during the dielectrophoresis, the dielectric particle 21 causes the positive precipitation into the first electric field region A by the action of positive dielectrophoresis (pDEP). On the other hand, when the real part of the Claudius-Mosotti coefficient takes a negative numerical value based on various conditions during the dielectrophoresis, the dielectric particle 21 causes the negative precipitation into the second electric field region B by the action of negative dielectrophoresis (nDEP).

Here, the frequency of the AC voltage at which positive dielectrophoresis and negative dielectrophoresis acting on the dielectric particle 21 are switched, namely the so-called cross-frequency, is different between the composite particle 31 and the unbound particle 32. Therefore, the composite particle 31, namely the dielectric particle 21 to which the target substance 11 is bound, can be separately precipitated by applying the AC voltage of a predetermined frequency to the electrode set 1111 based on the cross-frequency.

The predetermined frequency used here is (i) a frequency at which the positive dielectrophoresis acts on the composite particle 31 and the negative dielectrophoresis acts on the unbound particle 32 by the electric field gradient generated with the application of the AC voltage to the electrode set 1111, or (ii) a frequency at which the negative dielectrophoresis acts on the composite particle 31 and the positive dielectrophoresis acts on the unbound particle 32 by the electric field gradient generated with the application of the AC voltage to the electrode set 1111. Stated another way, the predetermined frequency is a frequency higher than a first frequency and lower than a second frequency. A set of the first frequency and the second frequency may be any one of a first set and a second set given below:
(1) First set=(first frequency=“frequency at which the positive dielectrophoresis acts on both the composite particle 31 and the unbound particle 32 by the electric field gradient generated with the application of the AC voltage to the electrode set 1111” and second frequency=“frequency at which the negative dielectrophoresis acts on both the composite particle 31 and the unbound particle 32 by the electric field gradient generated with the application of the AC voltage to the electrode set 1111”)
(2) Second set=(first frequency=“frequency at which the negative dielectrophoresis acts on both the composite particle 31 and the unbound particle 32 by the electric field gradient generated with the application of the AC voltage to the electrode set 1111” and second frequency=“frequency at which the positive dielectrophoresis acts on both the composite particle 31 and the unbound particle 32 by the electric field gradient generated with the application of the AC voltage to the electrode set 1111”)

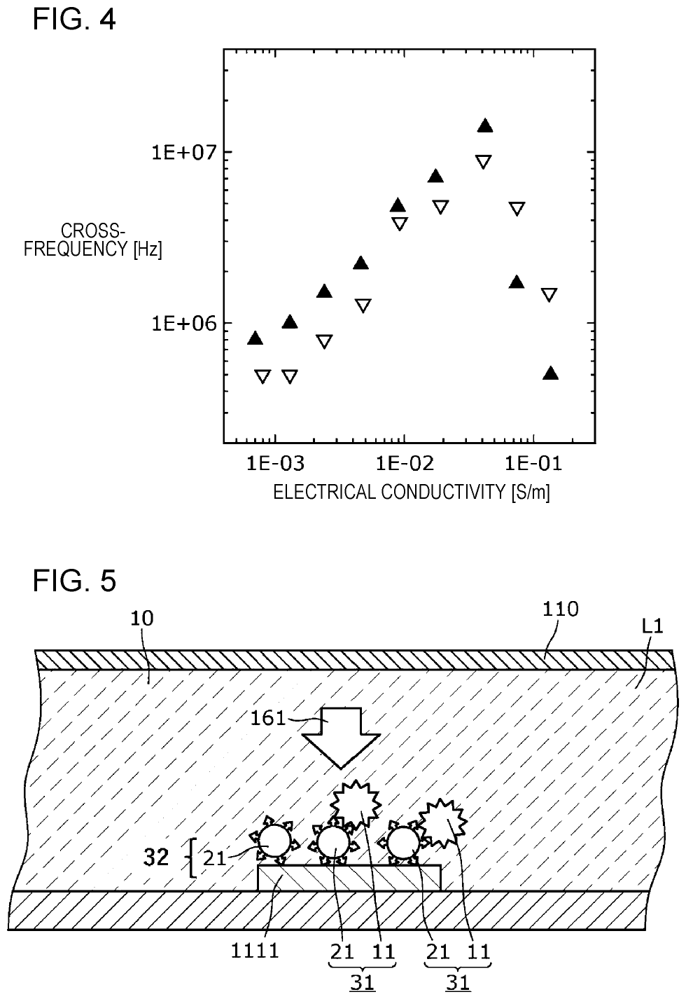

Additionally, the cross-frequency for the composite particle 31 and the cross-frequency for the unbound particle 32 are each variable depending on the electrical conductivity of the sample 10. FIG. 4 is a correlation graph between the cross-frequency for each of the composite particle 31 and the unbound particle 32 and the electrical conductivity of the sample 10 in the embodiment. In FIG. 4, the vertical axis indicates the cross-frequency (unit: Hz), and the horizontal axis indicates the electrical conductivity (unit: S/m) of the solvent. In FIG. 4, an upward black triangle represents data for the unbound particle 32, and a downward hollow triangle represents data for the composite particle 31.

As illustrated in FIG. 4, when the electrical conductivity of the sample 10 is lower than or equal to 0.03 S/m, the cross-frequency for the unbound particle 32 is higher than that for the composite particle 31. However, when the electrical conductivity of the sample becomes higher than or equal to 0.03 S/m, the cross-frequency for the composite particle 31 becomes higher than that for the unbound particle 32, and frequency characteristics are reversed. Accordingly, there may occur a phenomenon that, in spite of trying to separate and detect the composite particle 31, the unbound particle 32 is separated and detected. When the electrical conductivity of the sample 10 becomes higher than about 0.03 S/m, the cross-frequency starts to quickly reduce for each particle. Particularly, when the electrical conductivity of the sample 10 becomes higher than about 0.1 S/m, a sufficient dielectrophoresis force cannot be generated to act on each of the composite particle 31 and the unbound particle 32, and it becomes difficult to separate and detect the composite particle 31.

Thus, the electrical conductivity of the sample 10 is to be controlled to reduce. Taking the above point into account, in the detection method (the detection system 100) according to the embodiment, part or the whole of the first solvent L1 is replaced with the second solvent L2 with the lower electrical conductivity than the first solvent L1 depending on the cases such that the electrical conductivity of the sample 10 including the first solvent L1 reduces.

### Operation

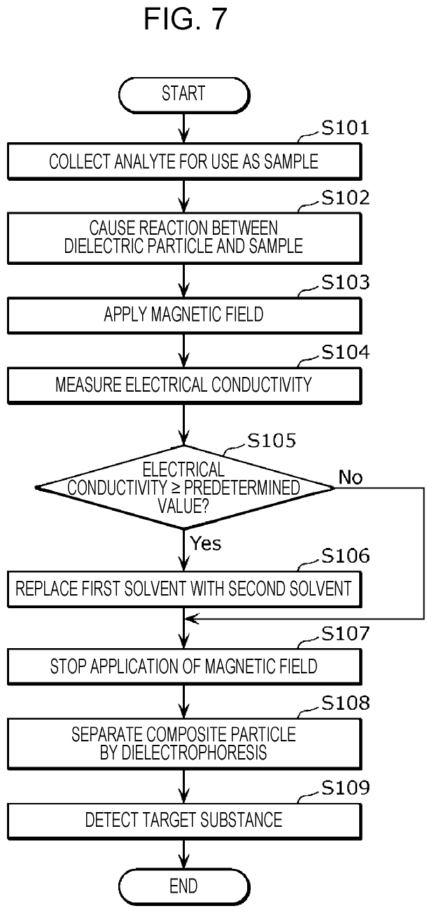

An example of the detection method (operation of the detection system 100) according to the embodiment will be described below with reference to FIG. 7. FIG. 7 is a flowchart representing the example of the detection method (operation of the detection system 100) according to the embodiment. Processes S101 and S102 described below are processes performed prior to starting to execute the detection method (operation of the detection system 100), and those processes may not need to be included in the detection method (operation of the detection system 100).

First, the analyte for use as the sample 10 is collected (S101). This process is executed with operation of an analyte collector (not illustrated). The analyte collector separates a fraction, supposed to include the target substance 11, from a fluid with a cyclone separator, a filter separator, or the like, thereby collecting the analyte to be detected. Alternatively, any of other known techniques for separating the fraction supposed to include the target substance 11, such as electrostatic collection, can also be optionally selected and applied to the analyte collector. Although depending on a configuration of the analyte collector, the fluid from which the fraction supposed to include the target substance 11 is to be separated may be a gas or a liquid. Stated another way, the detection system 100 can be applied to all kinds of targets by selecting the analyte collector corresponding to properties of the fluid. When a liquid fraction is obtained, the obtained fraction can be used as the liquid to prepare the sample 10 without adding any liquid to the obtained fraction. When a gaseous fraction is obtained, the obtained fraction is suspended into an aqueous solution, such as a phosphate-buffered physiological saline solution, and a resulting suspension is used as the liquid to prepare the sample 10. The liquid to prepare the sample 10 may also be called a liquid A.

Then, the sample 10 obtained by mixing the liquid A and the dielectric particle 21 is supplied to the space 1121. The liquid A and the dielectric particle 21 may be separately supplied to the space 1121 and mixed with each other in the space 1121. Upon the mixing of the liquid A and the dielectric particle 21, a binding reaction occurs (S102). When the liquid A includes the target substance 11, the composite particle 31 is formed. Accordingly, the sample 10 includes the composite particle 31 and the unbound particle 32 that is the dielectric particle 21 not bound to the target substance 11. The solvent included in the sample 10 is the solvent not replaced with the second solvent L2, namely the first solvent L1.

Then, the magnetic field 161 is applied to the sample 10 by operating the magnetic field applicator 160 (S103). With the application of the magnetic field 161, the composite particle 31 and the unbound particle 32 both included in the sample 10 are attracted to the electrode set 1111 by the magnetic field 161 and are retained in the vicinity of the electrode set 1111. While the sample 10 is introduced into the separator 110 once here, introduction and discharge of the sample 10 may be looped multiple times with the intent to retain, in the vicinity of the electrode set 1111, most of the composite particles 31 and the unbound particles 32 included in the sample 10.

Then, the measuring instrument 170 is operated to measure the electrical conductivity of the sample 10 including the first solvent L1 (S104). Here, if the measured electrical conductivity of the sample 10 is higher than or equal to the predetermined value (for example, 0.1 S/m) (S105: Yes), the exchanger 180 is operated to replace the solvent of the sample 10 from the first solvent L1 to the second solvent L2 (S106). With the replacement of the solvent, the electrical conductivity of the sample 10 is controlled to be held lower than the predetermined value. On the other hand, if the measured electrical conductivity of the sample 10 is lower than the predetermined value (S105: No), the exchanger 180 is not operated because there is no need of replacing the solvent of the sample 10 from the first solvent L1 to the second solvent L2.

After confirming that the measured electrical conductivity of the sample 10 is lower than the predetermined value, the application of the magnetic field 161 is stopped by stopping the operation of the magnetic field applicator 160 (S107). Then, the power supply 120 is operated and the AC voltage of the predetermined frequency is applied to the electrode set 1111, whereby the electric field is applied to the sample 10 to separate the composite particle 31 and the unbound particle 32 by the dielectrophoresis (S108).

Then, the detector 150 is operated, and the target substance 11 included in the composite particle 31 in the sample 10 is detected based on an image taken by the imager 140 (S109).

### Advantageous Effects and so On

As described above, the detection method according to the embodiment includes applying the magnetic field 161 to the sample 10 including the composite particle 31, the unbound particle 32, and the first solvent L1, thereby retaining the composite particle 31 and the unbound particle 32. Each of the composite particle 31 and the unbound particle 32 includes the dielectric particle 21 that has magnetism and is modified by the substance capable of specifically binding to the target substance 11. The composite particle 31 is bound to the target substance 11, and the unbound particle 32 is not bound to the target substance 11. The detection method according to the embodiment further includes, when the predetermined condition is satisfied, replacing at least part of the first solvent L1 with the second solvent L2 with the lower electrical conductivity than the first solvent L1 in the state in which the composite particle 31 and the unbound particle 32 are retained. The detection method according to the embodiment further includes stopping the application of the magnetic field 161 and applying the electric field, thereby separating the composite particle 31 and the unbound particle 32 by the dielectrophoresis. The detection method according to the embodiment further includes detecting the separated composite particle 31, thereby detecting the target substance 11.

With the above features, since at least part of the first solvent L1 is replaced, when the predetermined condition is satisfied, with the second solvent L2 with the lower electrical conductivity than the first solvent L1 in the state in which the composite particle 31 including the target substance 11 is retained, the electrical conductivity of the sample 10 (liquid) can be reduced while outflow of the composite particle 31 and the unbound particle 32 is suppressed. Thus, since the electric field is applied to the sample 10 with relatively low electrical conductivity, the advantageous effects are obtained in that it is easier to cause the sufficient dielectrophoresis force to act on the composite particle 31 and the unbound particle 32, to separate the composite particle 31, and hence to improve detection accuracy of the target substance 11.

Another method of applying a voltage to the electrode set 1111 to apply an electric field to the sample 10 including the first solvent L1 and to retain the composite particle 31 and the unbound particle 32 is also conceivable, by way of example, as a technique for replacing at least part of the first solvent L1 with the second solvent L2. In such a case, however, the electric field in the liquid tends to be weak, and only the composite particle 31 and the unbound particle 32 present in the vicinity of the electrode set 1111 can be retained, thus causing a problem that the composite particle 31 and the unbound particle 32 not present in the vicinity of the electrode set 1111 flow out at a high probability during the replacement of the solvent. It is here conceivable to, for increasing efficiency of retaining the composite particle 31 and the unbound particle 32, strengthen the electric field applied to the sample 10 including the first solvent L1. However, such a solution is not realistic from the viewpoint of safety.

By contrast, in the detection method according to the embodiment, since the magnetic field 161 is applied to the sample 10 including the first solvent L1, the advantageous effects are obtained in that the efficiency of the retaining the composite particle 31 and the unbound particle 32 can be increased in comparison with the case of applying the electric field to the sample 10 including the first solvent L1, and that the outflow of the composite particle 31 and the unbound particle 32 can be more easily suppressed during the replacement.

In the detection method according to the embodiment, the retaining the composite particle 31 and the unbound particle 32 is performed at the position of the electrode (the electrode set 1111) that applies the electric field to the composite particle 31 and the unbound particle 32.

Since the above feature increases a possibility that the composite particle 31 and the unbound particle 32 remain at the position of the electrode when the electric field is applied, the advantageous effects are obtained in that it is easier to cause the dielectrophoresis to act on the composite particle 31 and the unbound particle 32 and to separate the composite particle 31.

The detection method according to the embodiment further includes measuring the electrical conductivity of the sample 10. In the detection method according to the embodiment, the predetermined condition is that the electrical conductivity of the sample is higher than or equal to the predetermined value.

The above features provide the advantageous effects that the process of replacing at least part of the first solvent L1 with the second solvent L2 can be omitted when the electrical conductivity of the sample 10 including the first solvent L1 is lower than the predetermined value, and that the outflow of the composite particle 31 and the unbound particle 32 can be easily further suppressed.

In the detection method according to the embodiment, the dielectric particle 21 includes the magnetic particle.

The above feature provides the advantageous effect that the dielectric particle 21 can be more easily attracted by the magnetic field 161.

In the detection method according to the embodiment, the replacing the first solvent L1 with the second solvent L2 is performed by introducing the second solvent L2 to sweep away the first solvent L1.

The above feature provides the advantageous effect that the first solvent L1 can be easily replaced with the second solvent L2.

In the detection method according to the embodiment, the detecting the target substance 11 is performed by taking the image of the separated composite particle 31 with the imager 140 and by executing an image analysis of the taken image.

The above feature provides the advantageous effect that the target substance 11 can be easily detected.

The detection system 100 according to the embodiment includes the magnetic field applicator 160, the exchanger 180, the separator 110, and the detector 150. The magnetic field applicator 160 applies the magnetic field 161 to the sample 10 including the composite particle 31, the unbound particle 32, and the first solvent L1, thereby retaining the composite particle 31 and the unbound particle 32. Each of the composite particle 31 and the unbound particle 32 includes the dielectric particle 21 that has magnetism and is modified by the substance capable of specifically binding to the target substance 11. The composite particle 31 is bound to the target substance 11, and the unbound particle 32 is not bound to the target substance 11. The exchanger 180 replaces, when the predetermined condition is satisfied, at least part of the first solvent L1 with the second solvent L2 with the lower electrical conductivity than the first solvent L1 in the state in which the composite particle 31 and the unbound particle 32 are retained. The separator 110 stops the application of the magnetic field 161 and applies the electric field, thereby separating the composite particle 31 and the unbound particle 32 by the dielectrophoresis. The detector 150 detects the separated composite particle 31, thereby detecting the target substance 11.

With the above features, since at least part of the first solvent L1 is replaced with the second solvent L2 with the lower electrical conductivity than the first solvent L1 in the state in which the composite particle 31 including the target substance 11 is retained, the electrical conductivity of the sample 10 (liquid) can be reduced while the outflow of the composite particle 31 and the unbound particle 32 is suppressed. Thus, since the electric field is applied to the sample 10 with the relatively low electrical conductivity, the advantageous effects are obtained in that it is easier to cause the sufficient dielectrophoresis force to act on the composite particle 31 and the unbound particle 32, to separate the composite particle 31, and hence to improve the detection accuracy of the target substance 11.

### Modifications

The detection method and the detection system according to one or more aspects of the present disclosure have been described in connection with the embodiments, but the present disclosure is not limited to the above-described embodiments. Configurations obtained by variously modifying the embodiments in accordance with conceptions conceivable by those skilled in the art, and configurations obtained by combining the constituent elements in the different embodiments with each other may also fall within the scope of the one or more embodiments of the present disclosure as far as those configurations do not depart from the gist of the present disclosure.

While, in the embodiment, the electrical conductivity of the sample 10 including the first solvent L1 is measured, the present disclosure is not limited to that embodiment. For example, the first solvent L1 may be replaced with the second solvent L2 without measuring the electrical conductivity of the sample 10 including the first solvent L1, namely regardless of the value of the electrical conductivity of the sample 10 including the first solvent L1. In that case, the process of measuring the electrical conductivity of the sample 10 including the first solvent L1 may be omitted. Thus, in that case, the detection system 100 may not need to include the measuring instrument 170.

While, in the embodiment, the place where the magnetic field 161 is applied to the sample 10 including the first solvent L1 is the position of the electrode (the electrode set 1111), the present disclosure is not limited to that embodiment. For example, the place where the magnetic field 161 is applied to the sample 10 including the first solvent L1 may be different from the position of the electrode inside the space 1121.

While, in the embodiment, the electrical conductivity of the sample 10 including the first solvent L1 is measured in the state in which the magnetic field 161 is applied, the present disclosure is not limited to that embodiment. For example, the electrical conductivity of the sample 10 including the first solvent L1 may be measured prior to applying the magnetic field 161.

While, in the embodiment, the first solvent L1 is all replaced with the second solvent L2 by introducing the second solvent L2 to sweep away the first solvent L1, the present disclosure is not limited to that embodiment. For example, part of the first solvent L1 may be replaced with the second solvent L2 by skimming off a supernatant of the first solvent L1 and then pouring the second solvent L2.

A separation method can be realized by excluding the detection step from the detection method according to one aspect of the present disclosure, and such a separation method also falls within the aspects of the present disclosure.

In more detail, the separation method according to one aspect of the present disclosure includes applying a magnetic field to a sample including a composite particle, an unbound particle, and a first solvent, thereby retaining the composite particle and the unbound particle, each of the composite particle and the unbound particle including a dielectric particle that has magnetism and is modified by a substance capable of specifically binding to a target substance, the composite particle being bound to the target substance, the unbound particle being not bound to the target substance; replacing, when a predetermined condition is satisfied, at least part of the first solvent with a second solvent with lower electrical conductivity than the first solvent in a state in which the composite particle and the unbound particle are retained; and stopping the application of the magnetic field and applying an electric field, thereby separating the composite particle and the unbound particle by dielectrophoresis.

Furthermore, a separation system can be realized by excluding the detector from the detection system according to one aspect of the present disclosure, and such a separation system also falls within the aspects of the present disclosure.

In more detail, a separation system according to one aspect of the present disclosure includes a magnetic field applicator that applies a magnetic field to a sample including a composite particle, an unbound particle, and a first solvent, thereby retaining the composite particle and the unbound particle, each of the composite particle and the unbound particle including a dielectric particle that has magnetism and is modified by a substance capable of specifically binding to a target substance, the composite particle being bound to the target substance, and the unbound particle being not bound to the target substance; an exchanger that replaces, when a predetermined condition is satisfied, at least part of the first solvent with a second solvent with lower electrical conductivity than the first solvent in a state in which the composite particle and the unbound particle are retained; and a separator that stops the application of the magnetic field and applies an electric field, thereby separating the composite particle and the unbound particle by dielectrophoresis.

The present disclosure can be utilized as, for example, a detection system for detecting a target substance. such as a virus, which may cause an infectious disease.

What is claimed is:

1. A detection method comprising:
applying a magnetic field to a sample including a composite particle, an unbound particle, and a first solvent, thereby retaining the composite particle and the unbound particle, each of the composite particle and the unbound particle including a dielectric particle that has magnetism and is modified by a substance capable of specifically binding to a target substance, the composite particle being bound to the target substance, the unbound particle being not bound to the target substance;
replacing, when a predetermined condition is satisfied, at least part of the first solvent with a second solvent with lower electrical conductivity than the first solvent in a state in which the composite particle and the unbound particle are retained;
stopping the application of the magnetic field and applying an electric field, thereby separating the composite particle and the unbound particle by dielectrophoresis; and
detecting the separated composite particle, thereby detecting the target substance.

2. The detection method according to claim 1,
wherein the retaining the composite particle and the unbound particle is performed at a position of an electrode that applies the electric field to the composite particle and the unbound particle.

3. The detection method according to claim 1, further comprising:
measuring electrical conductivity of the sample,
wherein the predetermined condition is that the electrical conductivity of the sample is higher than or equal to a predetermined value.

4. The detection method according to claim 1,
wherein the dielectric particle includes a magnetic particle.

5. The detection method according to claim 1,
wherein the replacing the first solvent with the second solvent is performed by introducing the second solvent to sweep away the first solvent.

6. The detection method according to claim 1,
wherein the detecting the target substance is performed by taking an image of the separated composite particle with an imager and by executing an image analysis of the taken image.

7. A detection system comprising:
a magnetic field applicator that applies a magnetic field to a sample including a composite particle, an unbound particle, and a first solvent, thereby retaining the composite particle and the unbound particle, each of the composite particle and the unbound particle including a dielectric particle that has magnetism and is modified by a substance capable of specifically binding to a target substance, the composite particle being bound to the target substance, the unbound particle being not bound to the target substance;
an exchanger that replaces, when a predetermined condition is satisfied, at least part of the first solvent with a second solvent with lower electrical conductivity than the first solvent in a state in which the composite particle and the unbound particle are retained;
a separator that stops the application of the magnetic field and applies an electric field, thereby separating the composite particle and the unbound particle by dielectrophoresis; and
a detector that detects the separated composite particle, thereby detecting the target substance.
